(19) United States
(12) Patent Application Publication
(10) Pub. No.: US 2008/0051776 A1
(43) Pub. Date: Feb. 28, 2008

(54) Thin uninsulated cryoprobe and insulating probe introducer

(75) Inventors: Mordechai Bliweis, Kiryat-Haim (IL); Yaron Hefetz, Herzlia (IL)

Correspondence Address: Martin D. Moynihan;PRTSI, Inc., P.O. Box 16446, Arlington, VA, 22215, US

(73) Assignee: Galil Medical Ltd., Yokneam (IL)

(21) Appl. No.: 11/892,128

(22) Filed: Aug. 20, 2007

Related U.S. Application Data
(63) Continuation-in-part of application No. PCT/IL2007/000091, filed on Jan. 25, 2007.
Application No. PCT/IL2007/000091 is a continuation-in-part of application No. 11/637,095, filed on Dec. 12, 2006.
Application No. 11/637,095 is a continuation-in-part of application No. 10/660,478, filed on Sep. 12, 2003, now Pat. No. 7,150,743.
Application No. 10/660,478 is a continuation of application No. 09/860,486, filed on May 21, 2001, now Pat. No. 6,706,037.
Application No. 11/637,095 is a continuation-in-part of application No. 11/055,597, filed on Feb. 11, 2005.
Application No. 11/055,597 is a continuation of application No. 09/987,689, filed on Nov. 15, 2001, now abandoned.
Application No. 09/987,689 is a continuation of application No. 09/860,486, filed on May 21, 2001, now Pat. No. 6,706,037.
Application No. 11/637,095 is a continuation-in-part of application No. 11/185,699, filed on Jul. 21, 2005, now abandoned.
Application No. 11/185,699 is a division of application No. 10/151,310, filed on May 21, 2002, now abandoned.
Application No. PCT/IL2007/000091 is a continuation-in-part of application No. 11/640,309, filed on Dec. 18, 2006.
Application No. 11/640,309 is a continuation-in-part of application No. 10/660,478, filed on Sep. 12, 2003, now Pat. No. 7,150,743.
Application No. 10/660,478 is a continuation of application No. 09/860,486, filed on May 21, 2001, now Pat. No. 6,706,037.

(51) Int. Cl. A61B 18/02

(52) U.S. Cl. 606/21

(57) ABSTRACT

The present invention is of a cryotherapy apparatus comprising an uninsulated cryoprobe and an insulating introducer. Preferred embodiments include cryoprobes which are extremely thin and flexible because they lack an insulating layer and/or because their operating tip does not comprise a heat exchanger. Multi-cryoprobe introducers are provided.

### RELATED APPLICATIONS

This application is a continuation-in-part of PCT Patent Application No. PCT/IL2007/000091 filed Jan. 25, 2007, which is a continuation-in-part of pending U.S. patent application Ser. No. 11/637,095 filed Dec. 12, 2006, which is a continuation-in-part of U.S. patent application Ser. No. 10/660,478 filed Sep. 12, 2003, now U.S. Pat. No. 7,150,743, which is a continuation of U.S. patent application Ser. No. 09/860,486 filed May 21, 2001, now U.S. Pat. No. 6,706,037, which claims the benefit of U.S. Provisional Patent Application No. 60/242,455 filed Oct. 24, 2000, now expired.

U.S. patent application Ser. No. 11/637,095 is also a continuation-in-part of pending U.S. patent application Ser. No. 11/055,597 filed Feb. 11, 2005, which is a continuation of U.S. patent application Ser. No. 09/987,689 filed Nov. 15, 2001, now abandoned, which is a continuation-in-part of U.S. patent application Ser. No. 09/860,486 filed May 21, 2001, now U.S. Pat. No. 6,706,037, which claims the benefit of U.S. Provisional Patent Application No. 60/242,455, filed Oct. 24, 2000.

U.S. patent application Ser. No. 11/637,095 is also a continuation-in-part of U.S. patent application Ser. No. 11/185,699 filed Jul. 21, 2005, now abandoned, which is a divisional of U.S. patent application Ser. No. 10/151,310 filed May 21, 2002, now abandoned, which claims the benefit of U.S. Provisional Patent Application No. 60/300,097 filed Jun. 25, 2001, now expired, and U.S. Provisional Patent Application No. 60/291,990 filed May 21, 2001, now expired.

U.S. patent application Ser. No. 11/637,095 also claims the benefit of U.S. Provisional Patent Application No. 60/762,110 filed Jan. 26, 2006, now expired.

U.S. patent application Ser. No. 11/637,095 further claims the benefit of U.S. Provisional Patent Application No. 60/750,833 filed Dec. 16, 2005, now expired.

PCT Patent Application No. PCT/IL2007/000091 filed Jan. 25, 2007 is also a continuation-in-part of pending U.S. patent application Ser. No. 11/640,309 filed Dec. 18, 2006, which is a continuation-in-part of U.S. patent application Ser. No. 10/660,478 filed Sep. 12, 2003, now U.S. Pat. No. 7,150,743, which is a continuation of U.S. patent application Ser. No. 09/860,486 filed May 21, 2001, now U.S. Pat. No. 6,706,037, which claims the benefit of U.S. Provisional Patent Application No. 60/242,455 filed Oct. 24, 2000, now expired.

This Application is also being filed concurrently with U.S. National Phase patent application Ser. No. ______ filed xxxx, titled “DEVICE AND METHOD FOR COORDINATED INSERTION OF A PLURALITY OF CRYOPROBES” (Attorney Docket No. 31852).

This Application is also being filed concurrently with U.S. continuation-in-part (CIP) patent application Ser. No. ______ filed xxxx, titled “DEVICE AND METHOD FOR COORDINATED INSERTION OF A PLURALITY OF CRYOPROBES” (Attorney Docket No. 37192).

This Application is also being filed concurrently with U.S. continuation-in-part (CIP) patent application Ser. No. ______ filed xxxx, titled “DEVICE AND METHOD FOR COORDINATED INSERTION OF A PLURALITY OF CRYOPROBES” (Attorney Docket No. 37225).

The contents of all the above-mentioned applications are incorporated herein by reference.

### FIELD AND BACKGROUND OF THE INVENTION

The present invention relates to devices and methods for thermal ablation of a surgical target within a body of a patient. More particularly, the present invention relates to use of an introducer for delivering thermal ablation probes to an organic target, and to the design and use of very thin cryoprobes.

Cryoprobes cooled by Joule-Thomson cooling are a generally preferred form of cryoprobe in many clinical contexts. These are cryoprobes which cool by expansion of a high-pressure cooling gas such as argon to a low-pressure state, resulting in rapid cooling of the expanding gas. Cooling gas expansion typically takes place in an operating tip wherein gas from a high-pressure gas input lumen transits a Joule-Thomson orifice into an expansion chamber. As it enters the expansion chamber the cooling gas expands and cools, cooling the expansion chamber walls which then cool body tissues adjacent thereto.

To achieve the very low temperatures desirable for efficient cryoablation, such cryoprobes utilize a heat-exchanger (also referred to herein as a “heat exchanging configuration”) to pre-cool high-pressure cooling gas prior to expansion. Gas which is thus pre-cooled prior to expansion reaches extremely low gas temperatures after expansion. In typical prior art cryoprobes, a heat exchanger for pre-cooling is positioned to facilitate heat transfer from relatively warm (e.g. room temperature) high-pressure cooling gas supplied in a cryoprobe gas input lumen to very cold expanded cooling gas exhausting from the expansion chamber of the probe's operating tip and transiting the probe's gas exhaust lumen. Heat exchangers are typically constructed of highly thermally conductive materials such as metals and provide a large surface of contact between a gas input lumen and a gas exhaust lumen, to enhance thermal transfer from gas in one lumen to gas in the other. Various configurations are used to enhance thermal transfer, but the need to provide a large surface of contact generally results in relatively thick and bulky construction, thereby limiting thinness and flexibility of cryoprobes in which they are used.

U.S. patent application Ser. No. 11/651,997 by Ben-Zion Maytal, which is incorporated herein by reference, teaches an unusually thin cryoprobe providing various advantages in clinical use. Maytal's probe utilizes Krypton as a cooling gas, expansion characteristics of high-pressure krypton gas being such as to enable cooling to cryoablation temperatures without requiring a heat exchanger, resulting in probes which are significantly thinner than probes otherwise known to prior art.

### SUMMARY OF THE INVENTION

Embodiments of the present invention include an apparatus operable to deliver to a cryotherapy target a cryoprobe which is uninsulated, and therefore may be made thin and flexible, while providing thermal protection to healthy tissues positioned near a proximal shaft of that cryoprobe.

It is a disadvantage of many known prior-art cryoprobe designs that cryoprobe shafts containing conduits for exhausting cold cryogen fluid from the probe typically get so cold during probe operation as to endanger healthy tissues adjacent to the cryoprobe shaft. This danger, of tissues being damaged by proximity to proximal shafts of operating cryoprobes, is particularly acute when a cryoprobe shaft passes near an important or sensitive body region. Examples are a cryoprobe shaft passing near a neurovascular bundle when a probe operating tip is inserted in a portion of a prostate, a cryoprobe shaft passing through a cervix during fibroid treatment in a uterus, and a cryoprobe passing through cosmetically important skin during treatment of a breast. The problem is yet greater when a plurality of small probes is inserted in a common cryoablation target (as is desirable according to certain clinical protocols), causing a plurality of cryoprobe shafts also to be positioned close to one another and to be collectively positioned adjacent to portions of skin and other body tissues. For example, U.S. Pat. No. 6,142,991 to Schatzberger presents a system where templates are used to organize and control cryoablation of large lesions using multiple cryoprobes. However, it is a disadvantage of Schatzberger's system that use of his templates results in proximity of multiple cold cryoprobe shafts, which shafts tend to damage tissues near which they pass.

This problem is alleviated in prior-art cryoprobes by provision of an insulating layer which provides a thermal barrier between gas exhaust lumen and outer wall of a cryoprobe, thereby reducing thermal transfer between that gas exhaust lumen and body tissues near that lumen and adjacent to the shaft of the probe. Such an isolation layer, of course, necessarily adds thickness to the shaft of the probe, thereby increasing trauma to tissues through which a probe is inserted, and reduces flexibility of the probe, thereby limiting utility of cryoprobes in a variety of contexts. Thus, there is a widely recognized need for, and it would be highly advantageous to have, devices and methods enabling to deliver cryoprobes which are both thin and flexible to treatment targets, yet without endangering tissues proximate to the shafts of those probes.

The present invention successfully addresses the shortcomings of the presently known configurations by providing an apparatus operable to deliver a very thin and flexile probe to a cryotherapy target, while providing thermal protection to healthy tissues near a shaft of that probe. The present invention relates to use of a cryoprobe/introducer-sheath combination wherein a cryoprobe absent a thermally insulating layer is combined with a thermally insulating introducer. This apparatus can be used to extend towards and into a cryoablation target a cryoprobe operable to cryoablate portions of that target, which cryoprobe provides advantages of unusual thinness and high flexibility made possible by the fact that the cryoprobe itself does not comprise an insulating layer, yet the apparatus provides protection to healthy tissues by preventing unintended cooling by the cryoprobe shaft because the introducer comprises either a heater, or effective thermal insulation. In a method of use recommended for some embodiments, the introducer is inserted through a body lumen or into a body cavity and advanced toward a target tissue, whereupon a distal portion of the uninsulated probe is caused to extend distally from the introducer and is caused to penetrate into the target tissues. The cryoprobe is thus able to ablate target tissues while the introducer protects the body lumen and/or healthy tissues within the body cavity and/or all or most tissues external to the ablation target.

This combination of insulating introducer and uninsulated probe protects healthy tissue yet makes available a highly maneuverable probe able to penetrate tissue with a minimum of tissue resistance and tissue trauma. A thin ablation probe is advantageous in that it requires less force than a conventionally thick probe to penetrate tissue. This advantage is particularly important in certain clinical contexts, such as when a probe is required to penetrate a tough tissue such as a fibroid. Also, thin probes generally cause less trauma and bleeding than thick probes, when inserted into tissue. This, too, may be of critical importance in certain clinical contexts involving inserting a probe into very soft tissue. A liver, for example, is easily distorted or damaged when a non-thin probe is inserted therein by force.

The present invention further successfully addresses the shortcomings of the presently known configurations by reducing the complexity and manufacturing cost of cryoprobes and increasing their reliability. Cost, complexity, and potential for error in manufacturing cryoprobes having a thin insulation layer which must be finely fit within a narrow cryoprobe shaft are significantly greater than the cost, complexity and uncertainty of creating an uninsulated cryoprobe and an introducer, which may be a simple sheath, comprising material which is a poor heat conductor such as a plastic.

Treatment of uterine fibroids is an example of a clinical context where embodiments of the present invention may be advantageously used to treat ablation targets: an insulated introducer of the present invention may be used to deliver a thin cryoprobe to a fibroid, where thinness of the probe operating tip facilitates penetration of the fibroid, while insulating qualities of the introducer protect tissues of the cervix through which the cryoprobe must pass to reach the fibroid. In some embodiments, the cryoprobe operating tip may be formed as a spiral or other non-straight form, enabling to relatively large surface of contact between probe tip and target.

Cryotherapy is used to treat lesions in many parts of the body, yet some lesions which it would be desirable to treat using cryoablation or other forms of cryosurgery are not accessible to cryoprobes known to prior art. Some such lesions may be successfully treated by the cryoprobe/introducer combination of the present invention.

According to one aspect of the present invention there is provided a cryotherapy apparatus comprising a cryoprobe which comprises a treatment head coolable to cryoablation temperatures and a shaft having an external wall at least a portion of which cools to below 0° C. when the treatment head is cooled to the cryoablation temperatures; and an introducer insertable in a body of a patient, the introducer comprises a lumen sized to accommodate the cryoprobe, the introducer being adapted to prevent freezing of tissues adjacent to the introducer when a distal portion of the introducer is inserted in a body, the shaft wall portion is inserted within the distal introducer portion, and the treatment head is cooled to cryoablation temperatures. The introducer may comprise thermally insulating material and/or a heater such as an electric resistance heater. The cryoprobe may comprise a Joule-Thomson cooler or an evaporative cooler or other cooler. Preferably the cryoprobe is moveable within the introducer when the cryoprobe shaft portion is contained in the introducer and the introducer is inserted in a body, and the treatment head is distally extendable from the introducer when the introducer is inserted in a body. Preferably the treatment had is retractable into the introducer after having been distally extended from the introducer.

According to a further aspect of the present invention there is provided a cryotherapy apparatus comprising a cryoprobe introducer comprising thermally insulating material and having a lumen sized to accommodate a cryoprobe; and a cryoprobe having a distal treatment head coolable to cryoablation temperatures and a proximal shaft which comprises a cryogen input conduit, an external wall constructed of a homogeneous material, and a cryogen exhaust lumen defined between said cryogen input conduit and said external wall.

According to another aspect of the present invention there is provided a cryotherapy apparatus comprising an introducer and a cryoprobe. The introducer has a portion operable to be inserted into a body, the insertable portion comprises an external wall which comprises a tissue-protecting element selected from a group consisting of a thermally insulating material and an electric heater. The introducer further comprises a lumen sized to accommodate a cryoprobe, and a distal end. The cryoprobe comprises a distal operating tip operable to be advanced through the introducer lumen into an organic target within a body and to cool the target to cryoablation temperatures, and a proximal shaft having a shaft wall so designed and constructed that when the cryoprobe is inserted through the introducer and so positioned that the operating tip extends beyond the distal end of the introducer, less than 20% of that portion of the shaft wall which is then situated within the insertable portion of the introducer comprises effective thermal insulation.

Preferably, less than 5% of the portion of the shaft wall which is then situated within the insertable portion of the introducer comprises effective thermal insulation.

More preferably, less than 1% of the portion of the shaft wall which is then situated within the insertable portion of the introducer comprises effective thermal insulation.

In some embodiments, the shaft of the cryoprobe is entirely uninsulated.

According to further features in preferred embodiments of the invention described below, the cryoprobe is operable to be advanced and retracted within the introducer when a distal portion of the introducer is inserted in a body.

According to further features in preferred embodiments of the invention described below, the proximal shaft comprises markings showing position of the cryoprobe within the introducer. The markings may be calibrated to show by what distance a distal end of the cryoprobe extends beyond a distal end of the introducer.

According to still further features in preferred embodiments of the invention described below, cryoprobe and/or introducer comprise radio-opaque or ultrasound-visible markings, and imaging modalities are used to detect relative positions of probe and introducer as well as showing positions of both with respect to a therapeutic target or other anatomical landmarks.

In some embodiments the cryoprobe comprises a Joule-Thomson cryocooler.

The apparatus may comprise a positioning device for positioning the cryoprobe with respect to the introducer and a positioning sensor operable to report position of the cryoprobe with respect to the introducer. The apparatus may further comprise a thermal sensor.

According to further features in preferred embodiments of the invention described below, the tissue-protecting element is a heater, and the apparatus further comprises a controller for controlling the heater.

According to further features in preferred embodiments of the invention described below, the cryoprobe is a pre-bent cryoprobe.

According to further features in preferred embodiments of the invention described below, a distal portion of the lumen of the introducer is curved.

According to further features in preferred embodiments of the invention described below, the lumen of the introducer terminates on a side of the introducer, at a position proximal to a distal end of the introducer.

The apparatus may comprise a plurality of cryoprobes and the introducer may comprise a plurality of lumens. Some embodiments further comprise thermally insulating material between at least two of the lumens. Some embodiments do not comprise thermally insulating material between the lumens.

According to further features in preferred embodiments of the invention described below, the introducer lumen is sufficiently large to accommodate a plurality of cryoprobes.

According to further features in preferred embodiments of the invention described below, the cryoprobe comprises a relatively thin operating tip which comprises a Joule-Thomson orifice and an expansion chamber, and a relatively thick portion which comprises a heat-exchanger. In some embodiments the introducer comprises a relatively thick proximal portion sized to accommodate the relatively thick portion of the cryoprobe, and a relatively thin distal portion sized to accommodate the relatively thin operating tip.

According to further features in preferred embodiments of the invention described below, the introducer comprises an attaching device, such as for example a corkscrew-shaped hook, operable to attach the introducer to a therapeutic target.

According to a further aspect of the present invention there is provided a pre-bent therapeutic probe comprising a surface feature serving to orient the probe within a lumen of an introducer. The surface feature may be, for example, a ridge running along a length of an external wall of the probe.

According to a further aspect of the present invention there is provided an introducer having an internal lumen sized to accommodate a pre-bent therapeutic probe, which lumen comprises a surface feature operable to constrain a pre-bent therapeutic probe inserted therethrough to transit the lumen in a pre-determined orientation.

According to further features in preferred embodiments of the invention described below, the introducer comprises surface features operable to constrain a plurality of pre-bent probes inserted therein to diverge upon exiting from a distal end of the introducer.

According to a further aspect of the present invention there is provided a cryoprobe having a pre-bent distal end and a proximal handle operable to control orientation of the distal end when the cryoprobe is inserted in an introducer. Preferably, curvature of the pre-bent distal end and curvature of the handle are in a same plane.

According to a further aspect of the present invention there is provided a method for cryoablating an organic target, while protecting healthy tissue, comprising introducing a cryoprobe having a distal operating tip and a proximal shaft into an introducer which comprises a tissue-protecting element selected from a group consisting of a thermally insulating material and a heater, utilizing the introducer to deliver the operating tip to an organic ablation target, extending the tip from the introducer and inserting it in the target, and cooling the operating tip to cryoablation temperatures, thereby ablating the organic target, while the tissue-protecting element of the introducer prevents damage to healthy tissue by preventing destructive cooling of tissue adjacent the introducer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a and 1b are simplified schematics of Joule-Thomson cryoprobes according to the methods of prior art;

FIGS. 2a and 2b are simplified schematics of a cryotherapy apparatus comprising an uninsulated cryoprobe and an insulated introducer sheath;

FIGS. 3a, 3b, and 3c are simplified schematics of alternative embodiments of multi-probe introducers each operable to introduce a plurality of un-insulated cryoprobes into a body, according to embodiments of the present invention;

FIG. 4 is a simplified schematic of an un-insulated ultra-thin cryoprobe inside an insulating introducer, according to an embodiment of the present invention; and

FIG. 5 is a simplified schematic of a multi-probe introducer supplied with a plurality of pre-bent probes each with a handle, according to an embodiment of the present invention.

### DESCRIPTION OF THE PREFERRED EMBODIMENTS

The present invention relates to devices and methods for thermal ablation of a surgical target within a body of a patient. Specifically, the present invention can be used to deliver one or more uninsulated cryoprobes to an organic target, and to protect healthy tissues near proximal shaft portions of the cryoprobe, which shaft portions would otherwise risk cooling adjacent healthy tissue to damagingly cold temperatures when the cryoprobe operating tip is used for cryoablation. Cryoprobes constructed without thermal insulation in their shafts may be made thinner and more flexible than cryoprobes of prior art. An insulated introducer designed and constructed to penetrate into a body serves to deliver such cryoprobes to a treatment target. Once the introducer is appropriately positioned with respect to a target, an uninsulated probe inserted within that introducer may be freely advanced beyond a distal end of the introducer so that a distal operating tip of the inserted probe advances towards a target locus and is inserted as appropriate into target tissues. That distal operating tip is then cooled to cryoablation temperatures, thereby ablating target tissues. Thermal insulation comprised within the introducer prevents cold temperatures, induced in the cryoprobe shaft by cold expanded cooling gasses exhausting from the operating tip during cooling, from damaging tissues adjacent to the introducer.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrases “heat exchanger” and “heat-exchanging configuration” are used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice, referred to herein as a “Joule-Thomson orifice”, through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when passed through a Joule-Thomson heat exchanger. As is well known in the art, when gasses such as argon, nitrogen, air, krypton, CO₂, CF₄, and xenon, and various other gasses, at room temperature or colder, pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which, when passed at room temperature or warmer through a Joule-Thomson heat exchanger, have the property of becoming hotter. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating.

The terms “ablation temperature” and “cryoablation temperature”, as used herein, relate to the temperature at which cell functionality and structure are destroyed by cooling. According to current practice temperatures below approximately −40° C. are generally considered to be ablation temperatures.

The term “ablation volume”, as used herein, is the volume of tissue which has been cooled to ablation temperatures by one or more cryoprobes.

As used herein, the term “high-pressure” as applied to a gas is used to refer to gas pressures appropriate for Joule-Thomson cooling of cryoprobes. In the case of argon gas, for example, “high-pressure” argon is typically between 3000 psi and 4500 psi, though somewhat higher and lower pressures may sometimes be used.

The terms “thermal ablation system” and “thermal ablation apparatus”, as used herein, refer to any apparatus or system useable to ablate body tissues either by cooling those tissues or by heating those tissues.

For exemplary purposes, the present invention is principally described in the following with reference to an exemplary context, namely that of cryoablation of a treatment target by use of cryoprobes operable to cool tissues to cryoablation temperatures. It is to be understood that invention is not limited to that exemplary context. The invention is, in general, relevant to thermal treatment of any surgical target by means of one or more treatment probes delivered to that target through an insulating introducer. For simplicity of exposition, cryoprobes are presented in the Figures and reference is made to cryoprobes hereinbelow, yet all such references are to be understood to be exemplary and not limiting. Thus, discussion of cryoprobes hereinbelow may be understood to apply also to thermal probes of other sorts. Similarly, references to cryoablation of tissues are also to be understood as exemplary and not limiting. Thus, references to cryoablation are to be understood as referring also to non-cryogenic thermal ablation, and to non-ablative cryogenic treatment of tissues. Further, cryoprobes cooled by Joule-Thomson cooling are provided in examples presented by the Figures and discussed hereinbelow, yet it is to be understood that Joule-Thomson cryoprobes are presented for exemplary purposes only, and that selection is not to be understood to be limiting: references to Joule-Thomson cryoprobes are to be understood as referring as well to cryoprobes cooled by evaporative cooling, and to other cryoprobe embodiments. In particular it is noted that evaporative cryoprobes, in similarity to Joule-Thomson cryoprobes, often require shaft insulation to protect tissues near the cryoprobe shaft from damage by cold cryogen exhausting from a treatment head and flowing through a shaft of the cryoprobe, and it is to be understood that combinations of uninsulated evaporative cryoprobes together with insulating introducers are contemplated within the scope of the present invention.

It is expected that during the life of this patent many relevant cryoprobes and cryoprobe sheaths and cryoprobe introducers will be developed, and the scope of the terms “cryoprobe” and “sheath” and “introducer” is intended to include all such new technologies a priori.

As used herein the term “about” refers to ±10%.

As used herein and in the claims below, the term “substantially” refers to less more than 80%. Thus a statement that a portion of a cryoprobe shaft insertable into the body of a patient is “substantially uninsulated” implies that 80% or more of the surface area of that shaft does not comprise thermal insulating material.

In discussion of the various figures described hereinbelow, like numbers refer to like parts. The drawings are generally not to scale. Some optional parts may be drawn using dashed lines.

For clarity, non-essential elements are omitted from some of the drawings.

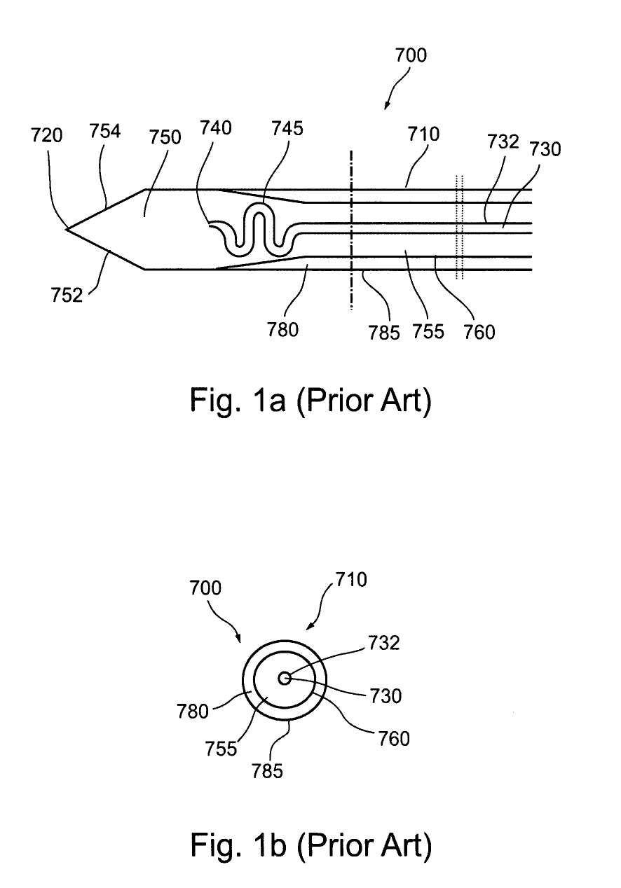

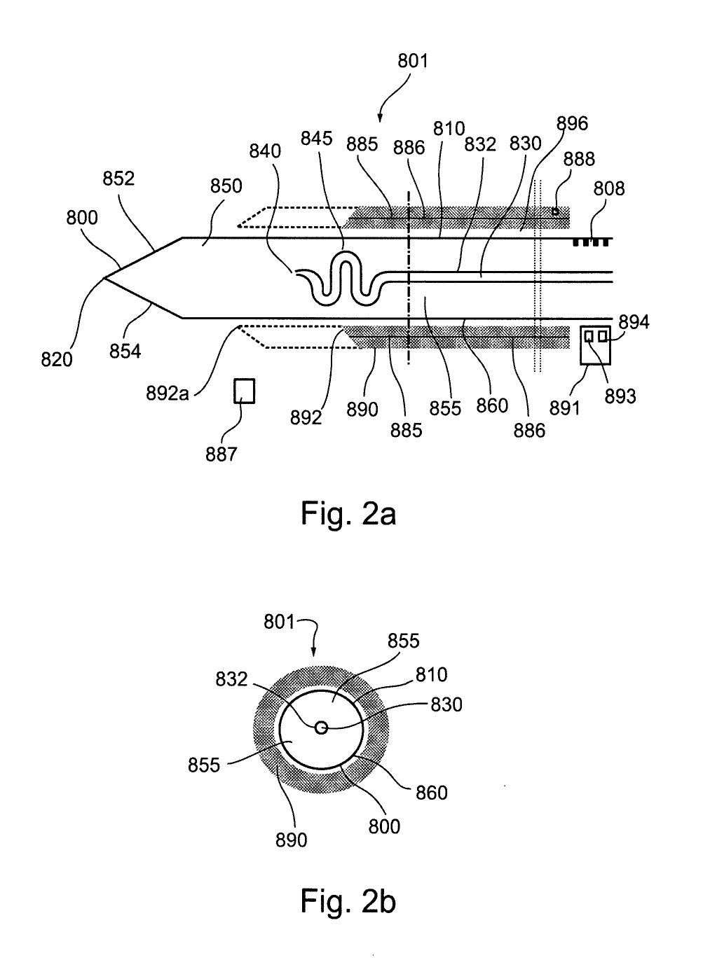

Attention is now drawn to FIGS. 1a and 1b, which are simplified schematics of Joule-Thomson cryoprobes according to methods of prior art, presented here for comparison with FIGS. 2a and 2b.

FIG. 1a presents a side-view cross-section of cryoprobe 700, which is a Joule-Thomson cryoprobe constructed according to the methods of prior art. Cryoprobe 700 comprises a shaft 710 having a sharpened distal end 720 used for penetrating tissue to be cryoablated. High-pressure cooling gas enters input gas lumen 730 of high pressure supply tube 732, passes through a heat exchanger 745 and exits through a Joule-Thomson expansion orifice 740 into an expansion chamber 750 within operating tip 754 of cryoprobe 700, where it expands. Expansion of cooling gas in chamber 750 cools the gas, and some gas may liquefy. External wall 752 of expansion chamber 750 is thereby cooled, and in turn cools body tissue surrounding operating tip 754. Heat absorbed from tissue surrounding operating tip 754 may causes liquefied gas within expansion chamber 750 (if any) to evaporate, the evaporation further cooling operating tip 754. Cold expanded and/or evaporated gas exhausts from expansion chamber 750 to atmosphere or to a gas collection system through a gas exhaust lumen 755. Gas exhaust lumen 755 is defined by high pressure supply tube 732 and wall 760 within shaft 710 of probe 700.

Cold gas exhausting from expansion chamber 750 flows over heat exchanger 745, thereby cooling high pressure cooling gas prior to arrival of that high-pressure gas at expansion orifice 740. Heat exchanger 745 is typically formed as a coiled tube, optionally with fins (not shown) serving to increase surface area through which heat is exchanged.

As explained above, shaft 710 will generally come in contact with healthy tissue which should not be ablated nor damaged. For example, a cryoprobe may be used for cryoablation of a fibroid. In this case, it is important to prevent freezing damage to the cervix through which cryoprobe 700 must be inserted to reach the fibroid. Similar protection may be useful when a cryoprobe is used to penetrate skin or other tissue in order to reach a target lesion to be treated.

It is noted that other embodiments of prior art cryoprobes, such as probes which cool by evaporative cooling rather than by Joule-Thomson cooling, similarly comprise shafts which contain gas exhaust conduits which similarly become cold when those probes' operating tips are active in cooling.

To avoid damage to healthy tissue, shafts of prior-art cryoprobes often comprise a layer of thermal insulation used to protect tissues adjacent to the shaft. Thus in FIG. 1a insulation layer 780 thermally insulates shaft 710, thereby protecting tissue adjacent to shaft 710 during cryoablation. Thermal insulation layer 780 may be created by a gap between wall 760 and outer wall 785 of shaft 710, which gap may be evacuated, or may be filled with gas of low thermal conductivity, or with another insulating material. Alternatively, wall 760 may be constructed of thermally insulating material, thereby forming insulator 780.

Light double dashed lines in FIG. 1a (and in the other Figures discussed hereinbelow) indicate optional absent portions of the apparatus, indicating that the apparatus may be considerably longer than shown in the drawings.

A heavy dashed line in FIG. 1a shows the location of a cross-sectional view seen in FIG. 1b. Thus, FIG. 1b presents a cross-sectional view of shaft 710.

As may be seen in FIG. 1b, high-pressure cooling gas enters probe 700 through input gas lumen 730 of high pressure tube 732. Cold expanded gas flows out of probe 700 to atmosphere or to a gas collection system through gas return lumen 755 defined by high pressure tube 732 and wall 760.

Thermal insulation layer 780 may be seen between gas return tube wall 760 and outer tube 785.

Attention is now drawn to FIGS. 2a and 2b, which are simplified schematics of a cryotherapy apparatus 801 comprising an uninsulated cryoprobe 800 and an insulating introducer sheath 890, according to an embodiment of the present invention.

FIG. 2a presents a side-view cross-section of apparatus 801. Uninsulated probe 800 is shown installed within a lumen of insulating introducer 890. Probe 800 is operable to be advanced and/or retracted within introducer 890 by a surgeon or other operator, or by an optional automatic positioning device 891 preferably comprising a remotely controlled displacer 893 such as a stepper motor, and/or a position sensor 894.

It is noted that FIGS. 2a and 2b present a cryoprobe 800 coolable by Joule-Thomson cooling. It is to be understood that the specific cooling technology of cryoprobe 800 as presented in FIGS. 2a and 2b is exemplary, and is not to be understood as limiting; cryoprobe 800 may be any cryoprobe operable to cool an operating tip to cryotherapy temperatures. A Joule-Thomson cryoprobe 800 is represented in FIGS. 2a and 2b for exemplary purposes, but cryoprobe 800 may be any cryoprobe. In particular, cryoprobe 800 may be a cryoprobe cooled by evaporation of a liquid.

As shown in exemplary FIGS. 2a and 2b, cryoprobe 800 comprises a shaft 810 having a distal end 820 which is preferably sharpened to facilitate penetration of body tissue. In similarity to probe 700 described above, when cryoprobe 800 is operated in cooling, high-pressure cooling gas enters input gas lumen 830 of high pressure tube 832, and passes through a Joule-Thomson orifice 840 into an expansion chamber 850 within an operating tip 854. Operating tip 854 is also referred to herein as treatment head 854.

In similarity to the process explained hereinabove with reference to cryoprobe 700, high-pressure cooling gas passing into expansion chamber 850 expands and thereby cools, and a portion may liquefy. External wall 852 of expansion chamber 850 is cooled by thermal contact with cooled expanded cooling gas within expansion chamber 850, and may also be cooled by evaporation of liquefied cooling gas therein. Cooled external wall 852 in turn cools body tissue surrounding operating tip 854. Cold expanded gas exhausts from expansion chamber 850 to atmosphere, or to a gas collection system, through gas exhaust lumen 855. Gas exhaust lumen 855 is defined by high pressure gas supply tube 832 and wall 860 of shaft 810 of probe 800.

Cold gas exhausting from expansion chamber 850 flows over heat exchanger 845, thereby cooling high pressure cooling gas therein prior to arrival of that high-pressure gas at expansion orifice 840. Heat exchanger 845 is typically formed as a coiled tube or similar configuration, optionally with fins (not shown) serving to increase surface area through which heat is exchanged.

In contrast to cryoprobes known to prior art, in a preferred embodiment of the present invention shaft 810 does not comprise an insulation layer. Instead, wall 860 is the outermost wall of shaft 810. In an embodiment of the present invention shown in FIG. 2a, wall 860 is thin, constructed of a homogeneous material such as stainless steel, and does not comprise insulating material. Thus, wall 860 both defines the outer limits of gas exhaust lumen 855 and constitutes the outer wall of shaft 810 of probe 800.

It is noted that, in some embodiments, for reasons of convenience certain portions of wall 860 may be insulating, but preferably most or all of wall 860, or in any case most or all of that portion of wall 860 designed for insertion into the body of a patient, is uninsulated and thereby may be made both very thin and very flexible. In a preferred embodiment, at least 80% of that portion of wall 860 designed for insertion into a body is substantially uninsulated, and that uninsulated portion is preferably thin and flexible.

Thus, in a preferred embodiment presented in FIG. 2a, apparatus 801 comprises a cryoprobe 800 which comprises a treatment head 854 coolable to cryoablation temperatures, and a shaft 810 having an external wall 860. Because wall 860 is substantially uninsulated and uninsulating, at least a portion wall 860 cools to below 0° C. when the treatment head is cooled to cryoablation temperatures. Apparatus 801 also comprises introducer 890 which is insertable in a body of a patient. Introducer 890 comprises a lumen 896 sized to accommodate cryoprobe 800. Since introducer 890 does comprise thermally insulating material (or, in alternative embodiments, a heater), introducer 890 is adapted to prevent freezing of tissues adjacent to introducer 890 when a distal portion of introducer 890 is inserted in a body, at least a portion of wall 860 of probe 800 is inserted within the inserted portion of introducer 890, and treatment head 854 of probe 800 is cooled to cryoablation temperatures.

As may be seen by comparing cryoprobe 700 with cryoprobe 800 as shown in FIGS. 1a-2b, absence in probe 800 of an insulation layer such as layer 780 of probe 700 enables significant reduction in the diameter of probe 800 as compared to probe 700.

A reduced-diameter probe 800 can be inserted more easily than a larger diameter probe 700 into body tissues, and will cause less trauma to those tissues when so inserted. A thinner probe 800 is also advantageous in that it may inserted into a body through a thinner working channel of an endoscope, for example a hysteroscope.

An additional advantage of cryoprobe 800 over probes known to prior art is that probe 800 may be made more flexible, again owing to lack of an insulation layer whose construction would add stiffness to the probe.

Probe 800 will also generally be easier to construct than probes of prior art having an insulating layer 780, and thus be made more cheaply than prior art probes. Alternatively, probe 800 may be constructed to be of higher quality and greater reliability than prior art probes at a similar manufacturing cost.

In an alternative to reduction in diameter, probe 800 may be constructed to be of diameter similar to diameters of prior-art probes 700, and yet present important advantages. In a probe 800 having a same outer diameter as a comparable probe 700, gas return lumen 855 of probe 800 may be made larger than corresponding gas return lumen 755 of probe 700, thus allowing higher vapor flow and reduced back-pressure in chamber 850 as compared to chamber 750. It is noted that in highly miniaturized cryoprobes preferred for many clinical uses today, miniaturization of the probe results in a restriction in the size of, and consequently in the gas flow within, gas exhaust lumens 755. Consequently, in typical use most miniaturized Joule-Thomson cryoprobes do not benefit from complete expansion (down to atmospheric pressure) of expanding cooling gas. Indeed, a back pressure in the neighborhood of 50 atmospheres may be measured in gas exhaust lumens 755 of typical miniaturized prior-art Joule-Thomson cryoprobes. The greater diameter of gas exhaust lumen 855 as compared to gas exhaust lumen 755 thus results in more complete expansion of cooling gas, resulting in a lower operating temperature of operating tip 854 as compared to that achievable by operating tip 754 in a probe 700 of comparable external diameter. Of course, lower achievable operating temperature constitutes an important advantage of cryoprobe 800 over prior-art probes 700.

Similarly, in a probe 800 having a same outer diameter as a prior art probe 700, input gas lumen 830 of probe 800 may be made larger than gas input lumen 730 of probe 700, thus allowing higher cooling gas flow, resulting in increased heat removal capacity of operating tip 854 as compared to that of operating tip 754.

Optionally, a combination of larger input gas lumen 830 and larger gas return lumen 855 may be used.

FIG. 2a shows cryoprobe 800 positioned within lumen 896 of introducer 890. Distal end 892 of introducer 890 is preferably sharpened to facilitate penetration of introducer 890 into body tissue. Distal end 892 of introducer 890 and distal end 820 of probe 800 may be formed to provide a relatively smooth and continuous distal surface when distal end 820 of probe 800 is positioned at distal end 892 of introducer 890. Thus in a preferred mode of operation distal end 820 of probe 800 is positioned at distal end 892 of introducer 890 prior to insertion of apparatus 801 into a body. A relatively continuous distal face of apparatus 801 thus created then facilitates insertion of apparatus 801 into body tissue. Alternatively, probe 800 may be inserted into introducer 890 after introducer 890 has already been positioned with its distal end near a cryotherapy target.

Sharp distal ends 820 and/or 892 may have a conical shape, a chisel or slanted shape similar to that typically used in hypodermic needles, or any other shape facilitating tissue penetration.

Alternatively, introducer 800 may have blunt or rounded distal end 892 appropriate for penetration into natural or man-made body cavity. A sharp cryoprobe 800 may be advanced from an unsharpened introducer 890 to be inserted into target tissue. Further alternatively, a blunt unsharpened cryoprobe 800 may be advanced out of an introducer 890 and used for cooling a tissue by applying thermal treatment to an accessible surface of that tissue, e.g. within a body cavity.

Introducer 890 preferably comprises material of low thermal conductivity. Introducer 890 thus serves to isolate cryoprobe shaft 810 from body tissues, and thereby protects those tissues when operating tip 852 is cooled to cryoablation temperatures and shaft 810 is cold. In a preferred embodiment insulation provided by introducer 890 is sufficient to protect body tissues proximate to introducer 890 during cryoablation procedures. Although it may sometimes be convenient for portions of shaft 810 to comprise insulation, in a preferred embodiment of the present invention most or all of shaft 810 is uninsulated. Specifically, if the term “insertable portion” refers to that portion of lumen 896 which is within a portion of introducer 890 sized and designed for insertion into a body, and the term “included portion” is used to refer to that portion of shaft 810 which is contained in an insertable portion of lumen 896 of introducer 890 when probe 800 is inserted through lumen 896 and positioned so that operating tip 854 extends beyond distal end 892 of introducer 890, then in a preferred embodiment of the present invention less than 20%, and more preferably less than 5%, and most preferably less than 1% of the included portion of wall 860 of shaft 810 comprises effective thermal insulation.

Thus, probe 800 may be constructed with little or no thermal insulation along its shaft 810, and introducer 890 serves to protect healthy tissue adjacent to introducer 890 during cooling of probe 800, by preventing those tissues from touching outer wall 860 of probe 800 and by reducing thermal transfer between tissues and outer wall 860 of probe 800.

In a preferred method of use, a surgeon positions introducer 890 so that distal end 892 is near a cryotherapy target, and advances cryoprobe 800 within introducer 890 so that a distal portion of probe 800, comprising operating tip 854 and optionally comprising a small portion of shaft 810, extends beyond distal end 892 of introducer 890. Thus, during cryoablation only a portion of probe 800 extending from distal end 820 of probe 800 to distal end 892 of carrier 890 is exposed (i.e. is without thermal insulation). The length of this exposed portion of probe 800 may be controlled by displacing introducer 890 relative to probe 800 or by displacing probe 800 relative to introducer 890, for example moving introducer 890 from the position marked 892 in FIG. 2a to that marked 892a on the Figure, or by moving it from position 892a to position 892, thereby respectively reducing or increasing the exposed portion of probe 800. Such a movement of introducer 890 relative to probe 800 (or equivalent movement of probe relative to introducer) varies the thermally exposed portion of probe 800 and thereby controls the heat removal capacity of probe 800. If introducer 890 is fixed in position relative to an organic target, advancing or retracting probe 800 relative to introducer 890 can also be used to control depth of penetration of probe 800 into that target.

In a preferred embodiment of the present invention, external wall 860 of shaft 810 of probe 800 comprises markings 808 visible to an operator, showing the position of probe 800 with respect to inserter 890. Markings 808 are preferably calibrated so as to indicate to an operator what length of distal portion of probe 800 extends beyond a distal end of introducer 890, when introducer 890 is inserted in a body of a patient and probe 800 is advanced within lumen 896 to such a position that operating tip 854 of probe 800 extends beyond distal end 892 of introducer 890.

As described above, introducer 890 preferably comprises thermal insulation which serves as a tissue-protecting element for protecting tissue adjacent introducer 890 from thermal damage during cryoablation. Optionally, introducer 890 may comprise, as an additional or alternative tissue-protecting element, a heater 885 to augment or replace thermal insulation for protection of healthy tissue. For example, as shown in FIG. 2a, an electric heater 886 constructed of thin electrical resistive wires may be integrated into introducer 890. A controller 887 may be provided to coordinate heating of heater 885 with cooling of probe 800 so as to maintain the temperature of the outer surface of introducer 890 within a range tolerable by surrounding tissue, for example between 0° and 42° C. Additionally or alternatively, one or more thermal sensors 888 may be provided within introducer 890, cryoprobe 800, or both. A thermal sensor 888 provided in introducer 890 may be used in a feedback loop to control heating of introducer 890.

A heavy dashed line in FIG. 2a shows the location of a cross-section view presented in FIG. 2b. FIG. 2b thus shows uninsulated shaft 810 of probe 800 positioned within insulating introducer 890. Gas exhaust lumen 855 is defined between high-pressure gas tube 832 and external wall 860 of probe 800.

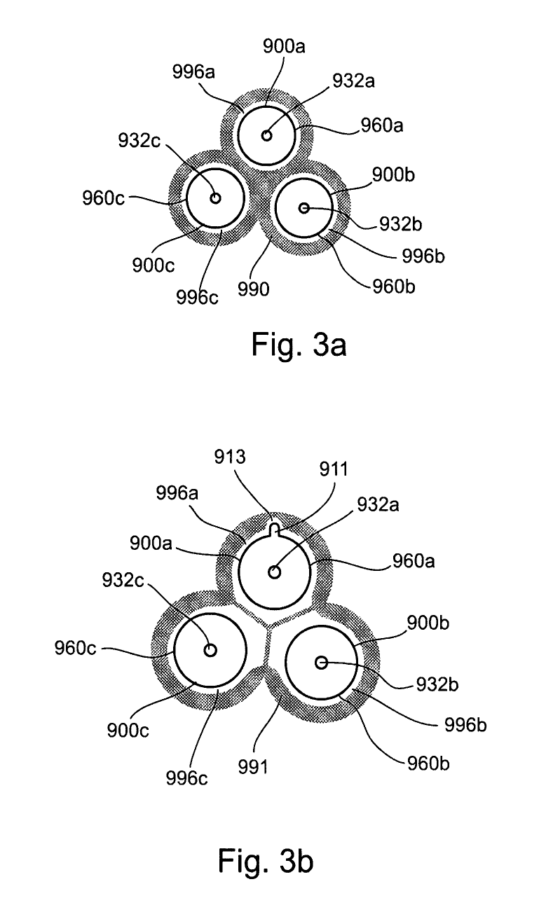

PCT Application IL2007/000091, incorporated herein by reference, teaches a variety of devices and methods using cryoprobe/introducer combinations to direct cryoprobes in pre-determined directions as they advance beyond introducers which deliver them to a vicinity of a therapy target. In particular, Application IL2007/000091 teaches use of a curved lumen to cause a distal end of a cryoprobe to acquire a lateral vector as it emerges from a distal end of an introducer. Lumen 896 of introducer 890 may by such a curved lumen. Application IL2007/000091 further teaches use of pre-bent probes operable to assume a curving form as they emerge from a distal end of an introducer. Probe 800 may be such a pre-bent probe. Further additionally, U.S. Pat. No. 6,706,037, also incorporated herein by reference, teaches use of introducer channels which terminate at a side rather than at a distal end of an introducer. Lumen 896 of introducer 890 (and/or one or more of lumens 996 discussed below with reference to FIGS. 3a-3c) may be such a side-terminating channel.

Attention is now drawn to FIGS. 3a, 3b and 3c, which are simplified schematics of multi-probe introducers each operable to introduce a plurality of uninsulated thermal probes into a body, according to embodiments of the present invention.

FIG. 3a presents multi-probe introducer 990 and a plurality of uninsulated probes 900. Three such probes, labeled 900a, 900b, and 900c, are shown in this exemplary Figure.

In similarity to FIG. 2b, gas input tubes 932a, 932b and 932c and outer walls 960a, 960b and 960c of probes 900a, 900b, and 900c respectively may be seen in FIG. 3a. Introducer 990, like introducer 890, comprises thermally insulating material and/or a heating element and serves to thermally isolate shafts of cryoprobes contained therein from tissues adjacent to introducer 990, as described hereinabove with respect to introducer 890. Introducer 990 is here presented with lumens 996a, 996b and 996c (each similar to lumen 896) for three probes, yet a smaller or larger number of lumens and probes may be used. Introducer 990 may be constructed with curved (e.g. distally diverging) lumens and/or may be used with pre-bent uninsulated probes.

FIG. 3b presents an alternative embodiment, wherein an introducer 991 is similar to introducer 990 of FIG. 3a, but differs therefrom in that thermal insulation is present only between probes 900 and tissues around introducer 990, no thermal insulation being presented between the several probes 900 within introducer 990. In other words, introducer 990 provides for probes 900 to be insulated from each other as well as from tissues outside introducer 990, whereas introducer 991 insulates shafts of probes 900 from body tissue, but not from each other. The configuration of introducer 990 is preferable in situations where at a given time one probe 900 may be used to cool tissue while another probe 900 is used to heat tissue, a situation which sometimes occurs in clinical practice. The configuration of introducer 991, on the other hand, is preferable when simultaneous heating and cooling is not contemplated, as introducer 991 has a smaller cross-section than introducer 990 (for a same size and number of cryoprobes) and will consequently penetrate body tissues more easily and inflict less trauma during penetration.

FIG. 3c presents yet another alternative configuration of a multi-probe introducer, labeled introducer 992. Introducer 992 is similar to introducers 990 and 991, yet does not provide individual channels for probes. Instead, introducer 992 provides a single large lumen sized to accommodate a plurality of uninsulated probes. Since no space within introducer 992 is taken up by internal subdivisions, introducer 992 presents an even smaller cross-sectional footprint than introducer 991 and is thus operable to penetrate body tissues even more easily and to inflict even less trauma during penetration. The external ‘clover-leaf’ form of introducers 990, 991 and 992 shown in FIGS. 3a-3c is exemplary only, and not limiting. In a preferred embodiment, introducer 992 in particular may be presented in cylindrical format, with probes 900 adjacent one another within a single internal lumen.

U.S. Patent Application IL2007/000091, discussed above, also teaches an introducer having an attaching device such as a corkscrew-shaped hook for attaching an introducer to an organic target during insertion of cryoprobes delivered to the target by the device. Introducers 990, 991 and 992 may comprise such an attaching device.

It is noted that probes 900a, 900b and 900c may be individually extended to varying controllable distances beyond the distal ends of introducers 990, 991, and 992, consequently heat removal capacities and lengths of target penetration of each probe may be individually controlled.

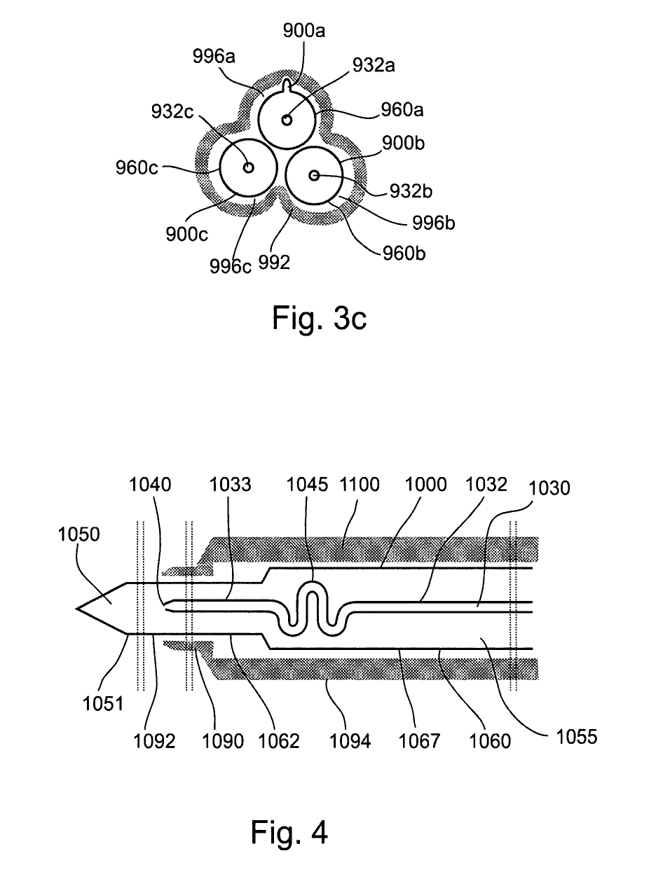

Attention is now drawn to FIG. 4, which is a simplified schematic of an uninsulated cryoprobe 1000 having an ultra-thin operating tip 1051, used in conjunction with an insulating introducer 1100, according to an embodiment of the present invention.

In order to further reduce the outer diameter of operating tip 1051 of cryoprobe 1000, a distal portion of cryoprobe 1000 comprises two sections: a thin (and optionally long) operating tip 1051 and a thicker section 1067 distinct from and optionally adjacent to operating tip 1051. Operating tip 1051 comprises a Joule-Thomson orifice 1040 and an expansion chamber 1050. Thicker section 1067 comprises a heat exchanger 1045.

In similarity to probe 800 and introducer 890 of FIG. 2a, probe 1000 comprises a gas input lumen 1030 within a high-pressure gas supply tube 1032 for supplying high pressure gas to heat exchanger 1045 and thence to Joule-Thomson orifice 1040. Also similarly, gas exhausts from expansion chamber 1050 by way of a lumen 1055 defined between outer wall 1060 and inlet gas tube 1032. As may be seen in FIG. 4, heat exchanger 1045, which is by nature relatively bulky because it serves to provide a large surface of contact between lumens 1030 and 1055, is positioned in thicker section 1067 of probe 1000, where there is room for it. In contrast, operating tip 1051, absent heat exchanger 1045, may be made extremely thin, as tip 1051 does not contain a heat exchanger and has no bulky parts.

Thermally insulating introducer 1100 can be used to deliver operating tip 1051 of probe 1000 to a cryotherapy target and to protect healthy body tissues near shaft wall 1060 of probe 1000 by thermally insulating those tissues from shaft wall 1060 during cryoablation.

Thus, cryoprobe 1000 comprises a relatively thin operating tip 1051 which comprises Joule-Thomson orifice 1040 and expansion chamber 1050, and a relatively thick portion 1067, (which may be either adjacent to or somewhat distant from operating tip 1051), which comprises heat-exchanger 1045. As may be seen in FIG. 4, introducer 1100 also preferably comprises thicker (i.e. larger diameter) and thinner (i.e. smaller diameter) portions, namely thicker proximal portion 1094 sized to accommodate portion 1067 of probe 1000, and a relatively thin distal portion 1090 sized to accommodate and allow passage of operating tip 1051. Thin distal portion 1090 of introducer 1100 may, however, be absent.

In contrast to embodiments depicted in FIGS. 1a and 2a, pre-cooled high-pressure cooling gas, after passing through heat exchanger 1045 where it is pre-cooled, passes further through a high-pressure conduit 1033 which transports pre-cooled high-pressure cooling gas from heat exchanger 1045 to expansion orifice 1040 located within thin (i.e. small diameter) operating tip 1051, which may be somewhat distant from heat exchanger 1045.

The pre-cooled cooling gas passes through orifice 1040 into expansion chamber 1050 within operating tip 1051, wherein it expands and further cools and may partially liquefy, cooling outer wall 1092 of operating tip 1051 and thereby cooling body tissues adjacent to that wall. Since heat exchanger 1045 is outside of operating tip 1051, operating tip 1051 can be manufactured having an extremely small diameter. Thinness of operating tip 1051 presents advantages of relatively easy and trauma-free penetration of tip 1051 into target tissue. Positioning of heat exchanger 1045 in larger section 1067 of cryoprobe 1000 provides room for a heat exchanger which is larger, more effective and more efficient than one which could possibly be provided within operating tip 1051. Thus, the configuration presented in FIG. 4 provides improved cryoprobe performance, enabling, for example, to achieve cryoablation temperatures with a relatively reduced flow of cryogen, and/or enabling to achieving lower tip temperatures than would otherwise be produced.

It is noted that probe 1000 may be moved relative to introducer 1100, thereby exposing more or less of operating tip 1051 beyond distal end 1090 of thermally insulating introducer 1100, thereby potentially controlling both length of penetration of operating tip 1051 into a target, and thermal performance characteristics of probe 1000.

Optionally, thicker portions of probe 1000 (portions containing bulky heat exchangers, for example) may be located in a handle of introducer 1100, or in other sections of introducer 1100 which do not penetrate into the body of a patient, or in any case which do not penetrate beyond body locations where larger diameter tubes may be tolerated.

Characteristics of introducer 1100 may be combined with those of multi-probe introducers 990, 991 and 992 presented hereinabove, to provide a cryosurgery apparatus operable to delivery to a cryotherapy target a plurality of cryoprobes each having an ultra-thin distal operating tip. Similarly, characteristics of introducer 1100 may be combined with those of introducer 890. Operating tip 1051 may be pre-bent, as described hereinabove.

Various physical lesions may be successfully treated using cryoprobe/introducer combinations similar to embodiments presented herein. Such situations include any which may be appropriately treated by means of an insulating sheath which delivers a probe part-way to an ablation target, protecting healthy tissues along the way, and from which a thin uninsulated probe may be extended from the sheath towards and/or into the target. For example, it may be found useful in some contexts to use such an introducer/probe combination to treat some cases of uterine fibroids, where one or more probes may be introduced into a uterus by means of an introducer which extends into and through a cervix, thereby protecting the cervix, and whence the probe or probes may be extended into the fibroid to perform fibroid ablation. For a same sharpness of tip, a very thin probe, such as is made possible by absence of insulation, will penetrate more easily into a fibroid than would an equivalent insulated probe, and would have a higher cooling capacity for a given diameter. Indeed, in a variety of contexts, then probes, so introduced, can more easily penetrate into tissues, cause less trauma during penetration, and reduce risk of bleeding.

Attention is again drawn to FIG. 3b, which shows an additional optional feature of introducer 991. In FIG. 3b, cryoprobe 900a may be seen to be provided with a ridge 911 sized to fit within a groove 913 provided in lumen 996a. Ridge 911 and groove 913 serve to constrain probe 900a to fit within lumen 996a with a predetermined orientation. It is to be understood that the particular configuration of groove and ridge presented in FIG. 3b is exemplary only, and not limiting: any appropriate combination of surface features constraining probe 900a to fit within lumen 996a in a particular orientation could be equivalently used. (For example, a groove might be provided in cryoprobe 900a and a ridge in lumen 996a.)

The groove-ridge combination, or other configuration constraining orientation of probe 900a within lumen 996a, is particularly useful if probe 900a is a pre-bent probe 912 as defined by U.S. Patent Application IL2007/000091 discussed hereinabove. Such orientation-constraining surface features may be utilized in any of the introducers presented herein, or in any other introducer for therapeutic probes, and will be particularly useful where pre-bent therapeutic probes of any sort are used. For example, a plurality of grooves in one lumen or in a plurality of lumens of an introducer may be used to constrain a plurality of pre-bent probes, each having a ridge fitting one of said plurality of grooves, to be positioned within said introducer in such orientations that distal portions of said pre-bent probes diverge as they advance beyond a distal end of that introducer.

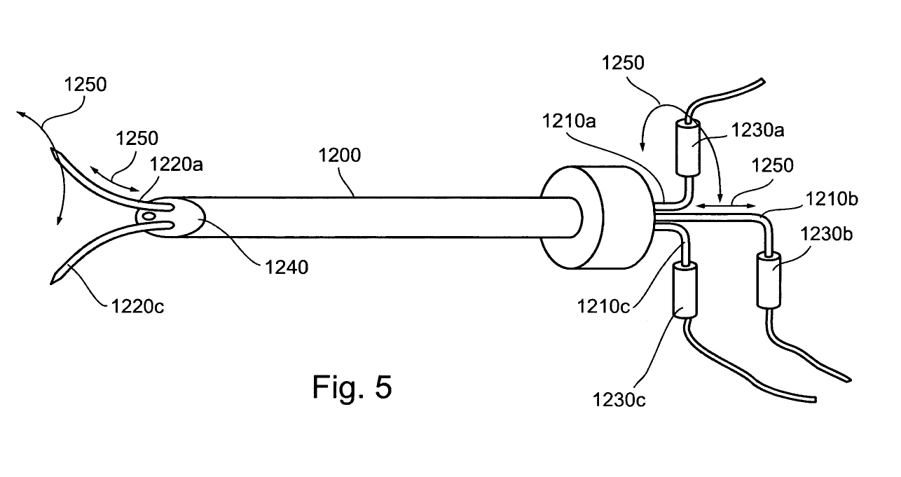

Attention is now drawn to FIG. 5, which is a simplified schematic of a multi-probe introducer supplied with a plurality of pre-bent probes with handles, according to an embodiment of the present invention. FIG. 5 demonstrates an additional method for controlling orientation of pre-bent probes advanced through an introducer. Probes 1210a, 1210b, and 1210c are shown inserted in introducer 1200. Probes 1210a, 1210b, and 1210c are provided with handles 1230a, 1230b, and 1230c respectively. Handles 1230 serve to show, by their position, the orientation of the pre-bent curves of probes 1210. (Handles 1230 are preferably curved in the same plane as the plane of curvature of the pre-bent distal ends of probes 1210, yet other orientations of handles 1230 are possible). Handles 1230 also serve to aid a surgeon in manipulating distal portions 1220 of probes 1210, as shown by movement arrows 1250. Note that distal portions 1220a and 1220c of probes 1210a and 1210c respectively may be seen extending from distal end 1240 of introducer 1200. Distal portion 1220b of probe 1210b is invisible, as it is retracted within introducer 1200.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A cryotherapy apparatus comprising a substantially uninsulated cryoprobe and a tissue-protecting introducer having a lumen sized to accommodate said cryoprobe.

2. The apparatus of claim 1, wherein said introducer comprises thermal insulation.

3. The apparatus of claim 1, wherein said introducer comprises an electric heater.

4. A cryotherapy apparatus comprising:
(a) a cryoprobe which comprises
(i) a treatment head coolable to cryoablation temperatures; and
(ii) a shaft having an external wall at least a portion of which cools to below 0° C. when said treatment head is cooled to said cryoablation temperatures; and
(b) an introducer insertable in a body of a patient, said introducer comprises a lumen sized to accommodate said cryoprobe, said introducer being adapted to prevent freezing of tissues adjacent to said introducer when a distal portion of said introducer is inserted in a body, said shaft wall portion is inserted within said introducer lumen, and said treatment head is cooled to cryoablation temperatures.

5. The apparatus of claim 4, wherein said introducer comprises thermally insulating material.

6. The apparatus of claim 4, wherein said introducer comprises a heater.

7. The apparatus of claim 4, wherein said cryoprobe comprises a Joule-Thomson cooler.

8. The apparatus of claim 4, wherein said cryoprobe is moveable within said introducer when said cryoprobe shaft portion is contained in said introducer and said introducer is inserted in a body.

9. The apparatus of claim 8, wherein said treatment head is distally extendable from said introducer when said introducer is inserted in a body.

10. The apparatus of claim 9, wherein said treatment had is retractable into said introducer after having been distally extended from said introducer.

11. A cryotherapy apparatus comprising
(a) a cryoprobe having
(i) a distal treatment head coolable to cryoablation temperatures; and
(ii) a proximal shaft which comprises a cryogen input conduit, an external wall constructed of a homogeneous material, and a cryogen exhaust lumen defined between said cryogen input conduit and said external wall; and
(b) a cryoprobe introducer comprising thermally insulating material and having a lumen sized to accommodate said cryoprobe.

12. A cryotherapy apparatus comprising:
(a) an introducer having a portion operable to be inserted into a body, said introducer comprises
(i) an external wall of said insertable portion which comprises a tissue-protecting element selected from a group consisting of a thermally insulating material and an electric heater; and
(ii) a lumen sized to accommodate a cryoprobe; and
(b) a cryoprobe which comprises
(i) a distal operating tip operable to be advanced through said introducer lumen into an organic target within a body and to cool said target to cryoablation temperatures; and
(ii) a proximal shaft having a shaft wall so designed and constructed that when said cryoprobe is inserted through said introducer and so positioned that said operating tip extends beyond said distal end of said introducer, less than 20% of that portion of said shaft wall which is then situated within said insertable portion of said introducer comprises effective thermal insulation.

13. The apparatus of claim 12, wherein less than 5% of said portion of said shaft wall which is then situated within said insertable portion of said introducer comprises effective thermal insulation.

14. The apparatus of claim 12, wherein less than 1% of said portion of said shaft wall which is then situated within said insertable portion of said introducer comprises effective thermal insulation.

15. The apparatus of claim 12, wherein said shaft of said cryoprobe is uninsulated.

16. The apparatus of claim 12, wherein said cryoprobe is operable to be advanced and retracted within said introducer when a distal portion of said introducer is inserted in a body.

17. The apparatus of claim 12, wherein said proximal shaft comprises markings showing position of said cryoprobe within said introducer.

18. The apparatus of claim 17, wherein said markings are calibrated to show by what distance a distal end of said cryoprobe extends beyond a distal end of said introducer.

19. The apparatus of claim 12, wherein said cryoprobe comprises a Joule-Thomson cryocooler.

20. The apparatus of claim 4, further comprising a positioning device for positioning said cryoprobe with respect to said introducer.

21. The apparatus of claim 4, further comprising a positioning sensor operable to report position of said cryoprobe with respect to said introducer.

22. The apparatus of claim 4, further comprising a thermal sensor.

23. The apparatus of claim 12, wherein said tissue-protecting element is a heater, and further comprising a controller for controlling said heater.

24. The apparatus of claim 4 wherein said cryoprobe is a pre-bent cryoprobe.

25. The apparatus of claim 4, wherein a distal portion of said lumen of said introducer is curved.

26. The apparatus of claim 4, wherein said lumen of said introducer terminates on a side of said introducer, at a position proximal to a distal end of said introducer.

27. The apparatus of claim 4, further comprising a plurality of cryoprobes.

28. The apparatus of claim 4, wherein said introducer comprises a plurality of lumens.

29. The apparatus of claim 28, further comprising thermally insulating material between at least two of said lumens.

30. The apparatus of claim 28, absent thermally insulating material between said lumens.

31. The apparatus of claim 4, wherein said lumen is sufficiently large to accommodate a plurality of cryoprobes.

32. The apparatus of claim 4, wherein said cryoprobe comprises a relatively thin operating tip which comprises a Joule-Thomson orifice and an expansion chamber, and a relatively thick portion which comprises a heat-exchanger.

33. The apparatus of claim 32, wherein said introducer comprises a relatively thick proximal portion sized to accommodate said relatively thick portion of said cryoprobe, and a relatively thin distal portion sized to accommodate said relatively thin operating tip.

34. The apparatus of claim 4, wherein said introducer comprises an attaching device operable to attach said introducer to a therapeutic target.

35. The apparatus of claim 34, wherein said attaching device is a corkscrew-shaped hook.

36. A pre-bent therapeutic probe comprising a surface feature serving to orient said probe within a lumen of an introducer.

37. The probe of claim 36, wherein said surface feature is a ridge running along a length of an external wall of said probe.

38. An introducer having an internal lumen sized to accommodate a pre-bent therapeutic probe, which lumen comprises a surface feature operable to constrain a pre-bent therapeutic probe inserted therethrough to transit said lumen in a pre-determined orientation.

39. The introducer of claim 38, further comprising surface features operable to constrain a plurality of pre-bent probes inserted therein to diverge upon exiting from a distal end of said introducer.

40. A cryoprobe having a pre-bent distal end and a proximal handle operable control orientation of said distal end when said cryoprobe is inserted in an introducer.

41. The cryoprobe of claim 40, wherein curvature of said pre-bent distal end and curvature of said handle are in a same plane.

42. A method for cryoablating an organic target, while protecting healthy tissue, comprising
(a) inserting a substantially uninsulated cryoprobe into an introducer;
(b) inserting said introducer into a body and advancing a distal portion of said introducer towards a therapy target;
(c) extending an operating tip of said cryoprobe from said distal introducer portion towards and into said therapy target;
(d) cooling said operating tip of said cryoprobe.

43. The method of claim 42, further comprising heating at least a portion of said introducer.

44. The method of claim 42, wherein said introducer comprises thermally insulating material.
